(19) United States
(12) Patent Application Publication
(10) Pub. No.: US 2026/0315304 A1
(43) Pub. Date: Oct. 15, 2026

(54) ATTACHMENT MECHANISM FOR POWERED LIMB ORTHOSES

(71) Applicant: CLEVELAND STATE UNIVERSITY, Cleveland, OH (US)

(72) Inventors: JERZY T. SAWICKI, Westlake, OH (US); JASON J. WIEBRECHT, Tallmadge, OH (US); JACOB A. STRICK, Mentor, OH (US); RYAN J. FARRIS, Dillsburg, PA (US)

(21) Appl. No.: 19/643,176

(22) Filed: Apr. 9, 2026

Related U.S. Application Data
(60) Provisional application No. 63/786,551, filed on Apr. 10, 2025.

(51) Int. Cl. A61H 3/00; A61F 5/01

(52) U.S. Cl. CPC A61H 3/00; A61F 5/0125; A61F 5/0127; A61H 2201/0107

(57) ABSTRACT

An attachment mechanism for a passive joint assembly is provided. The attachment mechanism allows a user to quickly transition from general, passive joint use to active, powered use as might be found in robot-assisted-gait-therapy (RAGT). In general, the devices and methods described herein provide means for removably attaching an active joint, thereby adapting a general use orthosis for active use. This enables a user to retain the benefits of their general use orthosis (e.g., custom-fit, smaller profile, and general familiarity with the device) with the powered orthosis in RAGT, with the added benefit of time savings by eliminating the time required to adapt/size the conventional powered gait therapy devices to each user.

### CROSS REFERENCE TO RELATED APPLICATIONS

This application claims priority to and the benefit of U.S. Provisional Application No. 63/786,551, filed Apr. 10, 2025. The content of which is hereby incorporated by reference in its entirety.

### TECHNICAL FIELD

The present disclosure is in the field of orthotics and more particularly in the field of powered limb orthoses.

### BACKGROUND

Leg injuries or disabilities, whether temporary or permanent, can affect a person's ability to walk, especially with a healthy gait. Knee-ankle-foot orthoses (KAFO) are orthotic devices designed to assist individuals with gait or lower limb injuries or disabilities achieve stable gait mechanics. Orthoses for day-to-day gait assistance/guidance are often customized to the individual user using thermoplastic or 3D printed cuffs that match the limb contour of the individual. These devices are often divided between the upper and lower portions of the limb (e.g., the knee joint in the case of the lower limb). The sections are connected by joint assemblies and supports made from steel or other sufficiently strong and rigid material to facilitate structured planar motion. When these individuals attend rehab or therapy (e.g., robot-assisted gait therapy (RAGT)), the current paradigm often requires these individuals to temporarily transition to orthoses that can provide guidance and powered assistance necessary for the therapy session. However, these RAGT devices largely consist of adjustable exoskeletons, which do not perfectly fit the user, are expensive to own/operate and have additional drawbacks that increase barriers to adoption.

The instant application provides solutions to address known drawbacks in current therapeutic systems.

### SUMMARY

The following is a brief summary of subject matter that is described in greater detail herein. This summary is not intended to be limiting as to the general inventive concepts or the scope of the claims.

The use of custom bracing in therapy is preferred, and no solution currently exists for the seamless integration of active joints (e.g., powered orthotic joints) with user-custom passive orthoses. A passive KAFO typically consists of thigh and shank cuffs connected by two joints located medial and lateral to the knee. The general inventive concepts enable the attachment of a robotic (or otherwise active) joint to a lateral passive joint in an existing orthosis and can be implemented in embodiments where the passive joint is modified or where the joint is replaced partially or entirely.

The general inventive concepts are based, in part, on the recognition that patient rehabilitation involving powered orthoses can be improved if the patient is able to use an orthosis that is more closely customized to their individual anatomy. Thus, the general inventive concepts contemplate an attachment mechanism for powered limb orthoses that enables a patient to utilize their customized general orthoses during e.g., robot-assisted gait therapy (RAGT) to receive and transmit gait power from a joint actuator, without needing to engage an entirely different orthotic device.

According to an exemplary embodiment of the present invention, an attachment mechanism is provided that enables adaptation of at least one joint of a patient's custom passive orthosis (i.e., general use orthosis) to receive and transmit powered limb movement for e.g., a therapeutic session. In certain exemplary embodiments, the attachment mechanism takes the form of one of two general categories, a passive joint modification or a passive joint replacement. Each of the passive joint modification and passive joint replacement retain a great many structural features of the patient's general use (passive) orthosis, while adapting the orthosis joint to receive powered limb torque to aid in rehabilitation.

In certain exemplary embodiments, the general inventive concepts contemplate a device for powered orthosis comprising a power source for providing gait power to a limb of an individual; an adapter for transmitting the gait power from the power source to a passive joint on an orthotic device on a limb of the individual, the orthotic device comprising a rotating member comprising first attachment feature, a non-rotating member comprising a second attachment feature, and a joint connecting the rotating member and the non-rotating member; the adapter comprising a housing, a first interface feature configured to releasably engage with the first attachment feature on the non-rotating member and a second interface feature configured to releasably engage with second attachment feature on the rotating member; such that gait power is transmitted from the power source to the joint.

In certain exemplary embodiments, the general inventive concepts contemplate a method of modifying a general use orthosis, the method comprising providing an adapter for transmitting the gait power from a power source to a passive joint on the general use orthotic device on a limb of the individual, the orthotic device comprising a rotating member comprising first attachment feature, a non-rotating member comprising a second attachment feature, and a joint connecting the rotating member and the non-rotating member; the adapter comprising a housing, a first interface feature configured to releasably engage with the first attachment feature on the non-rotating member and a second interface feature configured to releasably engage with second attachment feature on the rotating member; securing the adapter to the general use orthosis by engaging the first interface feature with the first attachment feature; engaging the second interface feature with the second attachment feature; and engaging the power source with the adapter to deliver the gait power to the passive joint.

### BRIEF DESCRIPTION OF THE DRAWINGS

These and other features of the present disclosure will become better understood with regard to the following description and accompanying drawings in which:

FIG. 1A is an image of an exemplary knee-ankle-foot orthosis.

FIG. 1B is an image of an exemplary knee-ankle-foot orthosis.

FIG. 1C is an example of a bilateral knee-ankle foot orthosis with an added trunk stabilization portion.

FIG. 2 is an image of a patient using a conventional general use knee-ankle-foot orthosis.

FIG. 3 shows 4 images of a conventional powered lower limb orthosis.

FIG. 4A shows an image of an orthotic with highlighting showing potential placements for attachment mechanisms for delivering powered torque to a patient (e.g., knee joints and ankle joints).

FIG. 4B shows an image of an orthotic with highlighting showing potential placements for attachment mechanisms for delivering powered torque to a patient (e.g., knee joints and hip joints).

FIG. 5A shows a general knee joint orthotic device.

FIG. 5B shows an orthotic device joint showing the interface between the non-rotating portion of the joint and rotating portion.

FIG. 6 shows an exemplary embodiment of a passive joint modification according to the general inventive concepts.

FIG. 7A shows an embodiment of an interface design for passive joint modification according to the general inventive concepts.

FIG. 7B shows an embodiment of an interface design for passive joint modification according to the general inventive concepts.

FIG. 7C shows an embodiment of an interface design for passive joint modification according to the general inventive concepts.

FIG. 7D shows an embodiment of an interface design for passive joint modification according to the general inventive concepts.

FIG. 8 shows an embodiment of a static interface for passive joint modification according to the general inventive concepts.

FIG. 9 shows an exemplary embodiment of a passive joint replacement according to the general inventive concepts.

FIG. 10 is an exploded view of a passive joint replacement device according to the general inventive concepts.

FIG. 11A shows a passive joint after replacement according to the general inventive concepts.

FIG. 11B is a cross-sectional view of the joint replacement of FIG. 11A, showing an embodiment with tooling to introduce a screw to fix the devices together.

FIG. 11C is a cross-sectional view of the joint replacement of FIG. 11A, showing an embodiment employing magnetic attachment to fix the devices together.

FIG. 12A is an image showing an adapted general use orthosis after attaching a powered, active joint thereto.

FIG. 12B shows an exploded view of an embodiment of an adapter according to the general inventive concepts.

### DETAILED DESCRIPTION

The following describes exemplary embodiments according to the general inventive concepts and is not intended to limit the scope of the claims in any way.

While certain exemplary embodiments described herein and illustrated in the drawings relate to an attachment mechanism for powered lower limb orthosis, it should be understood that many of the inventive features described herein may be applied to other devices, systems, and methods. For example, the features described herein may be utilized in other types of orthoses, including upper limb orthosis, or other electrically powered devices, including service robots or exoskeleton devices.

Various sections of the foregoing will refer to joints or portions of the relevant orthoses using human anatomical names (e.g., the knee joint, the upper leg/thigh portion). Those of ordinary skill will understand that these names are for ease of discussion and do not refer to the actual anatomical structures of the individual but are intended to refer to the areas or joints of the orthoses that rest against, stabilize, or facilitate the movement of the corresponding anatomical feature. Thus, the “knee joint” of an orthosis corresponds to the joint portion of the orthosis that is positioned near to the joint of the user when in operation and is designed/intended to stabilize a user's knee and to facilitate stable, predictable, planar movement thereof during a gait movement.

The term “powered orthosis,” “powered orthoses,” or “powered orthotic” as used herein, refer to an orthotic device that receives power from an external source (e.g., a cable drive) and translates the power to torque on e.g., the user's leg to facilitate movement of a joint. When discussing powered orthosis, it is important to understand that there is a wide range of applied power that might be required to aid individual subjects. For example, less power might be necessary to aid an individual if they need full assistance versus only mild or partial gait assistance. Likewise, larger individuals will require more torque than smaller individuals (e.g., adults versus children).

The term “passive joint” as used herein, refers to a joint on an orthotic device that does not receive power or torque from an external device and acts mainly as a stabilizer to enable the user to use the relevant joint(s) in a more stable manner during a gait movement. This may also be referred to herein as a joint on a general use orthotic device but may also encompass joints on powered orthoses that are designed to not receive power or do not currently receive power.

The term “active joint” as used herein, refers to a joint on a powered orthotic device that receives power or torque from an external device while providing stability to the user during their gait movement/cycle.

Various technologies pertaining to a device, system, and method for adapting general use orthoses, including attachment means to facilitate powered orthotic rehabilitation are discussed herein. In certain aspects, the general inventive concepts contemplate improved attachment for delivering powered torque to an individual at one or more predetermined joint(s).

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

As used herein, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

Ranges as used herein are intended to include every number and subset of numbers within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

Any combination of method or process steps as used herein may be performed in any order, unless otherwise specified or clearly implied to the contrary by the context in which the referenced combination is made.

Further, as used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary” is intended to mean serving as an illustration or example of something and is not intended to indicate a preference.

The general inventive concepts are based on the recognition that rehabilitation and associated activities can be improved for individuals undergoing gait therapy, including e.g., robot-assisted gait therapy (RAGT), if custom-fit devices are used to ensure optimal patient comfort and functionality. However, current solutions largely consist of adjustable exoskeletons, which do not perfectly fit the user, are expensive to own/operate and have additional drawbacks that increase barriers to widespread use/adoption. Individuals that participate in RAGT commonly wear custom-fit passive orthoses for day-to-day gait guidance. These orthoses contain thermoplastic or 3D printed cuffs that more specifically match the limb contour of the user, connected by strong, durable (e.g., steel) joint assemblies that facilitate structured planar motion. The general inventive concepts disclose various embodiments of an attachment mechanism designed to secure an active (i.e., powered, motor driven) joint to the knee joint of a passive knee-ankle-foot orthosis (KAFO), transforming the user-custom KAFO into a powered orthosis for RAGT. The mechanism allows for the quick connection of the active joint before a therapy session and quick disconnection afterward. In its most general form, the system includes a non-rotating member fixed to e.g., the thigh of a KAFO and a rotating member fixed to the shank of a KAFO. The inventive adapter mechanism enables the transmission of assistive torques from an actuator to the user. Generally speaking, an actuating device is configured to engage and move one or more components of the orthosis which causes a component to provide torque to the joint of the orthosis. This connection mechanism is versatile and can be used with upper and lower limb orthoses, with many transmission styles, including cable-driven, geared, belted and chained systems. Additionally, the attached joint does not need to be active; various passive joints can be attached to accommodate the specific needs of the patient.

In certain embodiments, the disclosed devices provide one or more of the following advantages: the attachment mechanism allows for the robotic actuation of user-custom orthoses; the attachment mechanism allows attachment and detachment to be done quickly to optimize user operating time during session; attachment installation can be made while system is worn on the body; mechanism does not necessarily require external tooling; mechanism is designed to be lightweight and low profile; the design prevents unintentional detachment; can be applied for robotic joints that provide controlled planar robotic assistance to one or more of the hips, knees, shoulders or elbows; can be applied to robotic exoskeletons are used in the following applications: industrial/military, mobility assistance, physical therapy; situations in which a quick attachment/detachment mechanism for a transmission would be beneficial.

Further, therapeutic sessions where the patient's needs are uncertain or changing could benefit from using multiple types of joints. The motor driven joint could be included in a kit alongside passive joints (e.g., a selectable range-limiting joint, a spring-loaded extension-bias joint, and a locked joint), allowing for easy swapping to best fit the user's needs. In practice, this concept would allow for the actuation of the passive orthosis worn by the user to the therapy session, instead of requiring them to use an entirely different device. Given the large financial costs associated with using a commercially adjustable exoskeleton, this concept presents a cost-effective solution for robot-assisted gait therapy.

Those of ordinary skill in the art will recognize that a range of force may be delivered to aid the patient in gait rehabilitation, as the range of applied torque varies depending on the individual's needs, anthropometrics, and the joint being actuated. For example, the average 11-year-old child requires approximately 30 Nm of hip/knee torque for full assistance, and exemplary cable-driven actuator (e.g., as shown in the examples) has a peak torque of 20 Nm, thus delivering partial assistance. For full ankle assistance in an average 11-year-old, approximately 50 Nm of torque is required. Thus, while the illustrated joint actuator has a torque rating of approximately 20 Nm; however, the required joint torque may vary depending on the application, as well as individual needs, user anthropometrics, and the specific joint being actuated. All of which should be interpreted to fall within the general inventive concepts unless expressly stated to the contrary or if it would conflict with generally understood principles associated with e.g., RAGT.

The present application is directed to an attachment mechanism for delivering movement torque to the joint of an individual to rehabilitate or assist a patient who is walking impaired. A lower limb orthosis is a device used to help assist, rehabilitate, and/or improve disorders of a person's lower limbs (e.g., a person's hip, elbow, or knee). In certain embodiments, an orthosis utilizing the attachment mechanism may be configured to provide assistive or rehabilitative torque to the lower limbs of the user.

An orthosis may include one or more actuating devices (also called “cassettes” or “cartridges”) that are configured to supply torque to the hip(s), knee(s), ankle(s), or any combination of the hip(s), knee(s), and ankle(s) of a user. In certain embodiments, the actuating device aids in rehabilitating a user's gait by guiding the user's walking to a healthy gait. In addition, the actuating device may be configured to assist a walking user by decreasing the user's necessary exertion on one or more limbs of the user. In an exemplary embodiment, the actuating device may be configured to aid and assist the gait of the walking impaired pediatric population (i.e., children between the ages 6 and 11). However, the actuating device may be used in several other applications that require the described features, such as, for example, providing performance improving torque to the lower limbs of healthy individuals, as a joint actuator in an upper limb orthosis where quiet operation is needed, as a joint actuator in service robots where quiet operation is needed, or any other suitable application.

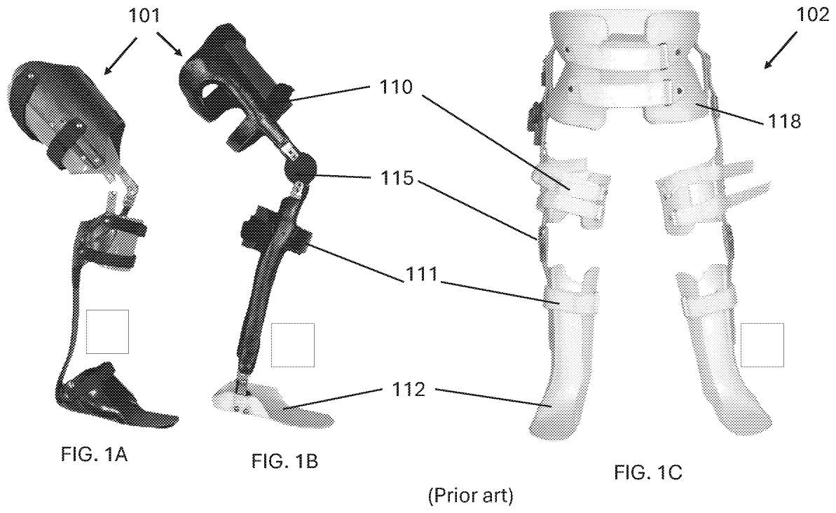

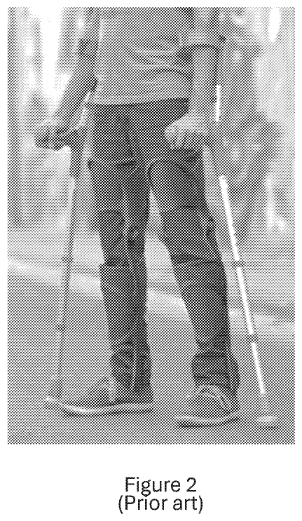

FIG. 1 shows several embodiments of conventional lower limb orthoses, two knee-ankle-foot orthoses (KAFO) 101, and a third orthosis (102, also known as a hip-knee-ankle-foot orthosis)) that includes harnessing and support for the trunk of the user 118, and lateral support joints for the hips of the user. Each of the orthoses include upper limb/thigh cuffs 110 for securing the KAFO to the thigh/upper portion of the limb, lower leg cuffs (also called a shank cuff) 111 for securing the KAFO to the lower portion/shin of the user's limb, and foot/ankle support 112 for the user's feet. Each of the orthoses includes at least one knee joint 115, that constrains joint movement in a planar direction via the vertical support members that run along the user's leg. The knee joint will include a rotating member and a non-rotating member. FIG. 2 shows an image of a conventional passive or general use KAFO similar to those shown in FIG. 1 as worn by a user.

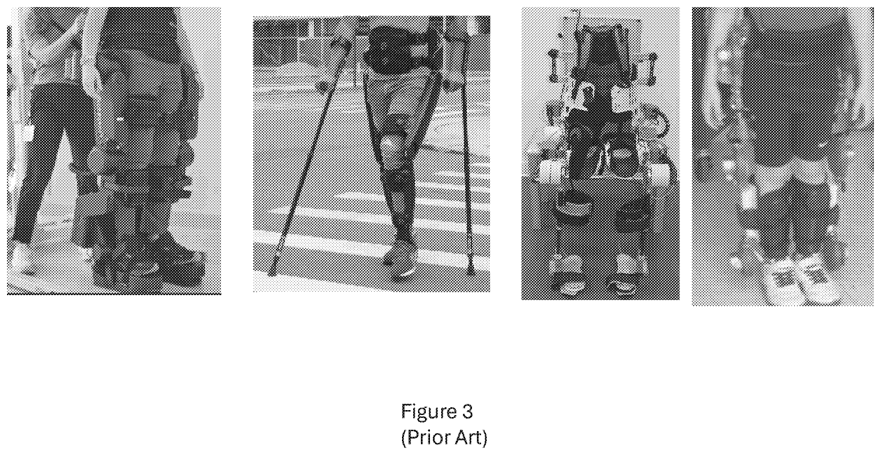

FIG. 3 shows several images of conventional KAFO that include various robot-assisted or otherwise powered movement (e.g., active orthoses). As can be seen from the images, each of these conventional robot-assisted-gait-therapy embodiments are considerably bulkier than the passive system shown in FIG. 2 and are less customized/tailored to the individual user's shape. In practice, this can lead to less effective or efficient rehabilitation sessions and require considerable effort and time on the part of the therapist to “size” the active system to each user at the onset of the therapy session.

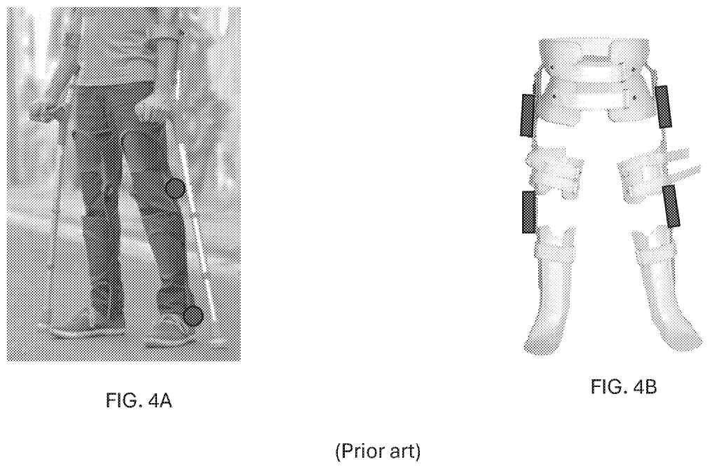

FIG. 4A shows the passive orthosis system of FIG. 2, including highlighted placement areas where the attachment mechanism of the general inventive concepts could be positioned/engaged to provide active torque to at least one of the KAFO joints. Locations include the knee joint and the ankle joint for this particular embodiment. FIG. 4B shows an alternative embodiment based on the passive orthosis system of FIG. 1C, showing attachment locations on the hip joint and at the knee joint of the orthosis.

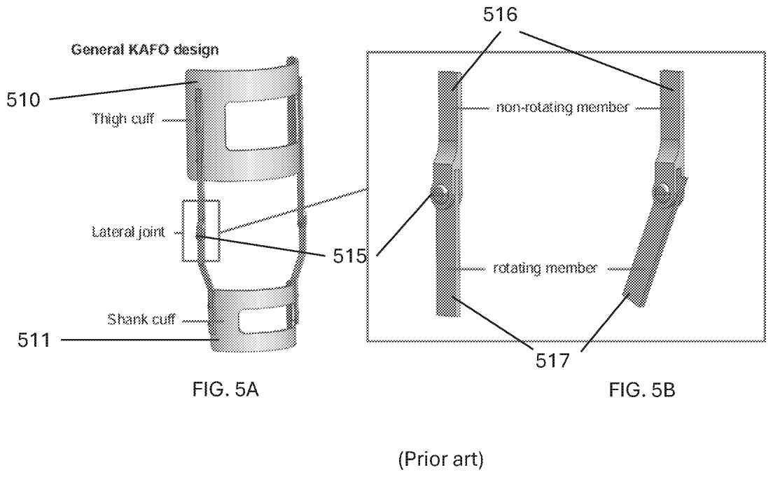

FIG. 5A shows a knee-joint portion of a conventional orthotic with a thigh cuff 510, shank cuff 511, and lateral joint (or knee joint) 515 (5A shows two joints located medial and lateral to the knee, the general inventive concepts will be discussed herein showing attachment of the power delivery device on only the lateral joint side of the KAFO, though one of ordinary skill in the art will recognize that embodiments wherein the attachment is positioned medially would also fall within the general inventive concepts). FIG. 5B is a close-up view of the joint of FIG. 5A, showing the upper/thigh support member 516 that would be attached to the thigh cuff and the lower/shank support member 517, that would be attached to the shank cuff. The joint, upper support member, and lower support member will generally be made from a rigid material, often metal, that can deliver sufficient strength to both constrain and support the user's joint during the gait cycle. The image on the left side of 5B shows the orthosis wherein the joint is extended whereas the right side of the image shows the joint during rotation of the (knee) joint. While the terms “rotating member and non-rotating member” may be used to describe the portions of the support structure, those of ordinary skill in the art will recognize that each of the pieces will be required to move during the individual's motion during gait rehabilitation or any other use of the device. The terms “rotating” and “non-rotating” are not intended to be limiting but rather, reflect distinctions in how the individual parts move relative to the joint in question during joint usage. In other words, during routine usage, the upper or non-rotating member is more stationary relative to the individual's body, whereas the lower/rotating member is expected to move more during the gait cycle of the individual. Those of ordinary skill in the art will also recognize that various other arrangements of the parts are possible, while maintaining the intended stabilization and planar movement of the relevant joint.

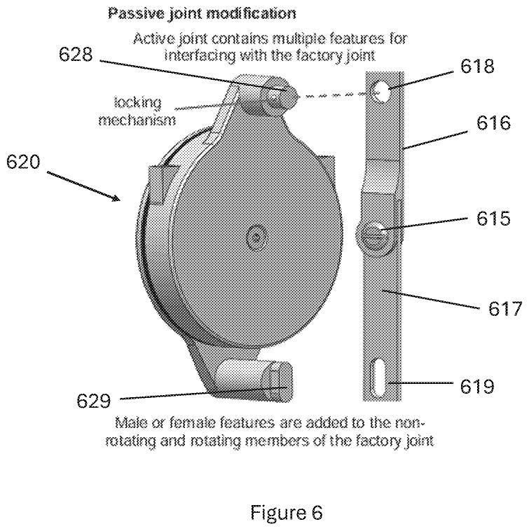

FIG. 6 shows an exemplary embodiment of a passive joint modification according to the general inventive concepts. In the embodiments shown, the passive joint includes at least one connecting feature (here shown as apertures to accept connections from the active joint) on both the rotating and non-rotating support members to ensure adequate torque transmission. At least one of these features should incorporate a locking mechanism to secure the active joint to the general use joint, preventing unintended disassembly during use. The interfacing features between the active and passive joints can be located on the support members (as shown in FIG. 6). The locking features may or might not require external tooling and can consist of several mechanisms. Four locking features are shown below, all of which are designed for the modification embodiment and do not require external tooling. Since these mechanisms do not rely on external tooling, they may be preferred in situations where multiple joints will be swapped in a therapy session.

In FIG. 6, the joint 615, upper support member 616, and lower support member 617, are similar to those shown in FIG. 5, but in the embodiment shown in FIG. 6, the lower support member and upper support member each include features for mating/engaging/interfacing with a power-delivery device 620. The upper support member includes a first attachment feature 618 (shown here as a circular aperture). While the general inventive concepts contemplate a variety of shapes and sizes of the first aperture, when the first aperture is a part of the non-rotating portion of the joint, it may be preferable if the aperture is circular, as shown in FIG. 6, as the circular shape allows freedom of planar motion/rotation between the power-delivery device and the joint. This facilitates necessary motion during the gait movement. The first attachment feature 618 is sized and designed to interface with a first interface feature 628 on the adapter (power-delivery device). As can be seen from the figure, the first interface feature 628 has a cylindrical size/shape designed to mate/engage with the first attachment feature 618, and also includes a locking mechanism (here, shown as a locking pin) to hold the power-delivery device and the joint together and maintain the interface between the devices. The lower support member includes a second attachment feature 619 (shown as a generally elliptical shaped aperture). In a similar fashion to the first attachment feature, the second attachment feature is designed and sized to mate with a second interface feature 629 of the power-delivery device. Those of ordinary skill in the art will recognize that a variety of different aperture/interface shapes combinations are conceivable that remain within the general inventive concepts, such as square, star, cross, and rectangular shapes, among others. As can be seen from the figure, the second interface feature 629 (here shown as a protrusion extending outward from an arm on the lower end of the adapter housing) has a generally elliptical shape designed to mate with the second attachment feature 619. While FIG. 6 shows the first and second attachment features as apertures and the corresponding interface features as corresponding protrusions extending from the power-delivery device, the general inventive concepts contemplate embodiments wherein the passive joint includes protrusions extending from the individual support members that are sized and shaped to interface with corresponding apertures on the power-delivery device, essentially swapping the features shown in FIG. 6 from the power-delivery device to the passive joint. In certain exemplary embodiments, at least one of the first attachment feature and the second attachment feature have a shape that constrains the relative motion of the power-delivery device once it is interfaced with the orthosis. More specifically, the shape of the second aperture and the second interface feature are designed to prevent relative rotation for delivery of the power/torque to the joint for powered orthosis. The shape of the upper interface and that of the lower interface should have a different shape or profile such that a user cannot inadvertently attach the power delivery device in an incorrect orientation (e.g., mistakenly attaching the device upside down). In the embodiment shown in FIG. 6, the first aperture and first interface feature have a circular/cylindrical shape that enables relative motion (rotation) between the orthosis and the power-delivery device, whereas the second aperture/interface feature does not allow movement on that portion of the interface, allowing for torque in the joint to power the user's joint movement.

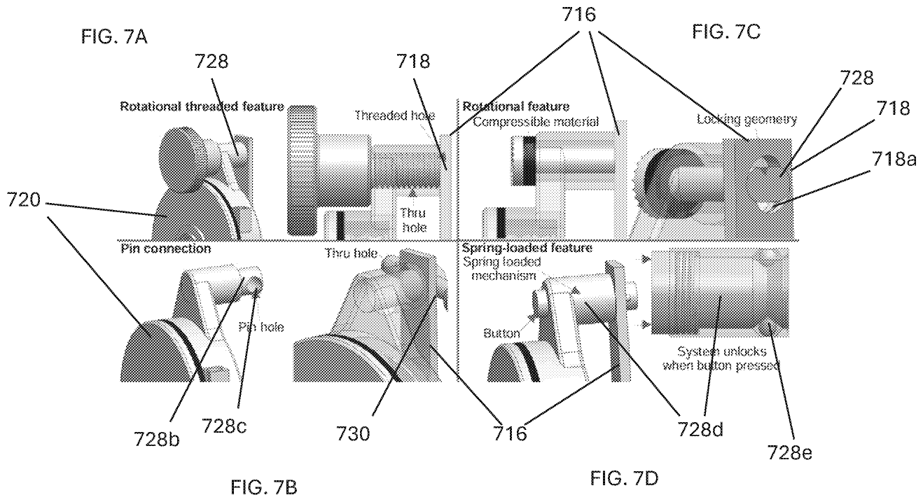

FIG. 7A-D shows various non-limiting embodiments for interfaces between the passive joint and the adapter 720, including various embodiments of locking mechanisms, in joint modification according to the general inventive concepts. FIG. 7A shows an exemplary embodiment wherein a first/top interface feature 728 includes a threaded portion 728a and a corresponding first/top attachment feature 718 (that includes a corresponding threaded aperture) on the (non-rotating) upper support member 716. FIG. 7B shows an embodiment wherein the interface feature 728 includes a pin hole 728c formed through a protruding cylinder extending from the end of an arm 728b. The interface feature is designed with a sufficient length such that the pin hole is accessible on the opposite side of the joint member 716 when engaged thru the attachment feature on the support member (here, shown as the upper/non-rotating member), allowing a pin 730 to be inserted into the pin hole, attaching the power-delivery device to the orthosis. FIG. 7C shows an arrangement wherein the interface feature includes a compressible material and a locking geometry between the interface feature 728 and the attachment feature 718 (here shown as an aperture with locking geometry shape). As can be seen from the figure, the arm of the interface feature is inserted into the attachment feature and rotated. A channel 718a within the support member of the orthosis mates with a flange (not shown) on the arm of the interface feature. Upon rotation of the interface feature (arm) 728, the flange constrains the relative motion, and the compressible material delivers counterforce into the interface, securing the two together. FIG. 7D shows an embodiment wherein the interface feature includes a button-activated spring-loaded interface mechanism (also referred to as a quick-release pin with spring loaded balls). In this embodiment, the support member of the orthosis includes a channel within the support that is sized to accept ball bearings. The interface unlocks when a button on the interface feature is pressed, thereby disengaging the ball bearings, allowing the interface arm to be removed from the aperture in the orthosis member.

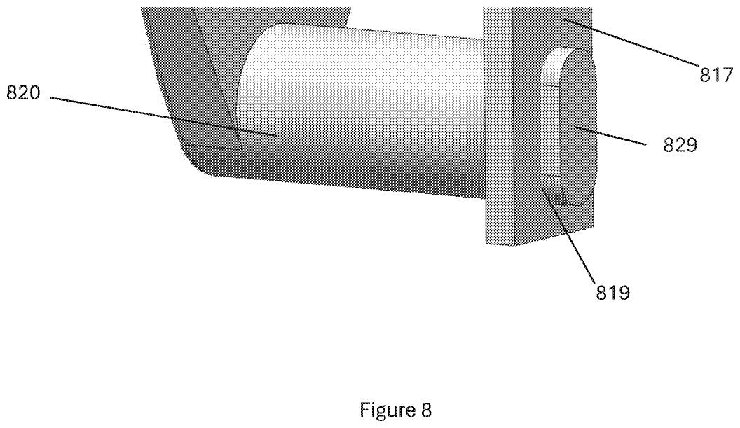

FIG. 8 is a close-up view showing an embodiment of the second interface feature 829 extending from an arm of the adapter housing engaged/mated with an embodiment of the second attachment feature 819 on an orthosis support member 817.

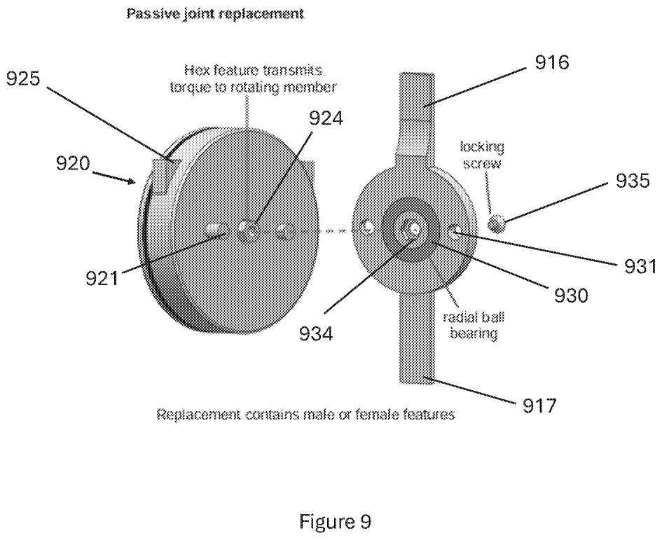

FIG. 9 shows an exemplary embodiment of a passive joint replacement system. The passive joint replacement may include a radial movement member (e.g., radial ball bearings) 930 positioned between the non-rotating (upper support 916) and rotating (lower support 917) members. In the embodiment shown in the figure, the inner diameter of the radial movement feature (radial ball bearings) is press-fit to the rotating (lower support) member, while the outer diameter is slip-fit to the non-rotating (upper support) member. The interfacing features between the active and passive joints can be located on the axis of rotation (as shown in the Figure), as opposed to being located on the support members. The upper support member 916, includes attachment features 931 (here shown as apertures through the member), which correspond in a mating relationship to interface features 921 on the power delivery device 920. The active joint also includes a torque delivery feature 924 (shown as a hex head with a threaded hole in the center) which can serve the dual purposes of delivering torque to the joint and providing a locking mechanism when a fastener 935 such as a screw is engaged from the opposite side of the joint. The torque delivery feature is inserted in a mating relationship with a correspondingly shaped receiving feature 934 on the passive joint. While the figure shows a six-sided hex arrangement, those of ordinary skill in the art will recognize that other shapes and corresponding receiving features are possible while still falling within the general inventive concepts. The passive joint replacement may incorporate range-of-motion hard stops to prevent unnatural movement of the anatomical joint. During passive use, a component secures the joint assembly, which is removed and replaced with the robotic joint during active use (e.g., during RAGT).

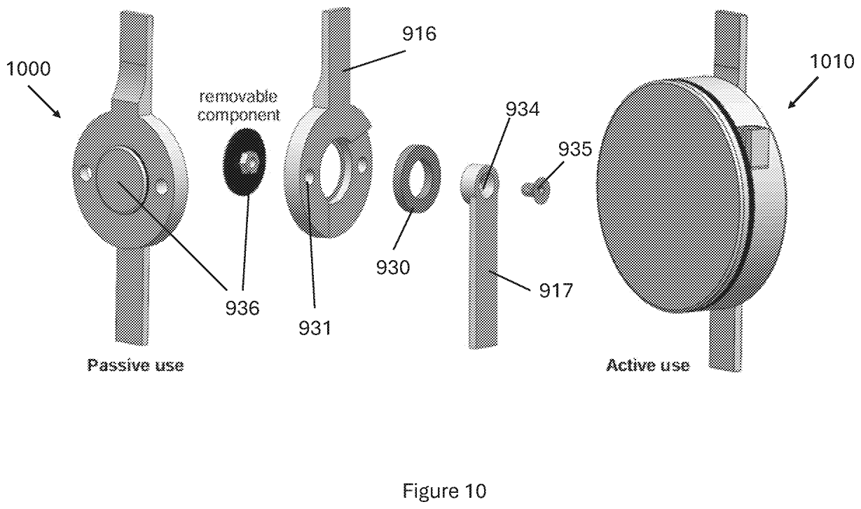

FIG. 10 shows an embodiment of a joint in passive use 1000 (with corresponding exploded view) and in active use 1010 after passive joint replacement. The passive use joint includes a removeable fastening member 936 which engages with the fastener 935 when engaged through the receiving feature 934. The figure shows the radial movement feature 930 (e.g., ball bearings) and the upper support member 916 and lower support member 917. In certain embodiments, the attachment features 931 on the upper support member may not be engaged during passive or general use of the orthosis.

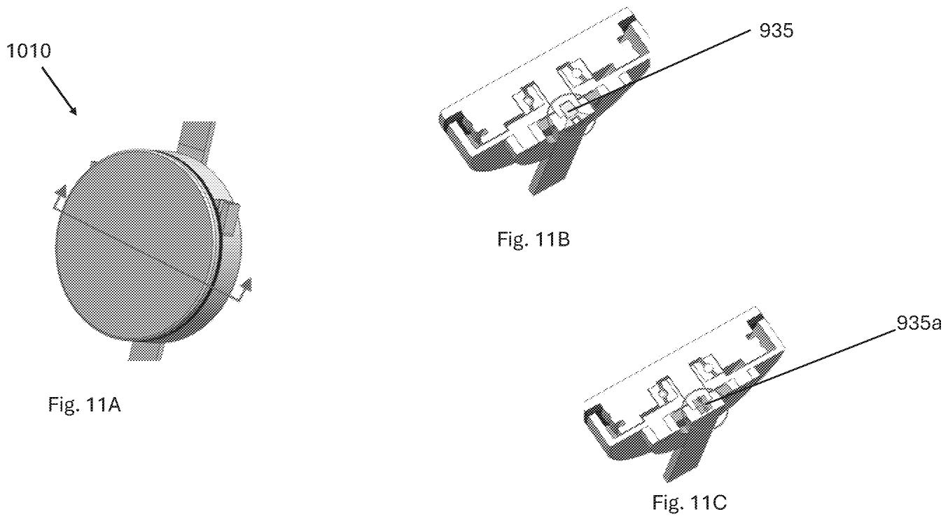

FIG. 11A shows an embodiment of an active joint assembly 1010. FIG. 11B shows an embodiment of an active joint assembly similar to that shown in FIG. 10 with a screw fastener 935 highlighted in the circle, for removably attaching the active joint to the user's orthosis. FIG. 11C shows an alternative embodiment wherein the screw fastener is replaced with a magnetic coupling 935a attaching the active joint housing to the user's orthosis. Additional locking mechanisms requiring external tooling might include a bolt and nut or snap rings. Locking mechanisms that do not require external tooling might include a quick-release buckle or latch. Connecting features without locking capabilities are designed to withstand planar loads. All concepts incorporate various geometric cutouts, such as circles, squares, hexagons or slots. Below, a male/female slot connecting feature is shown.

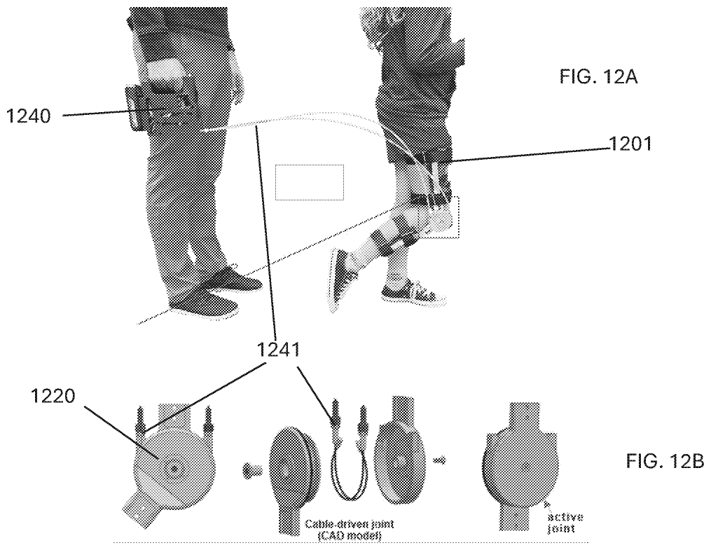

FIG. 12A shows an embodiment with an active joint 1220 engaged with the user's orthosis at the knee joint with a cable driven power source 1240 providing torque to the joint during gait movement (e.g., RAGT). FIG. 12B also shows an exploded view of the interior features of a cable-driven active joint 1220, showing the power-delivery cables 1241 exiting the adapter through holes in the housing. While the embodiment shown in FIG. 12 is cable-driven, the power source for the active joint may include a variety of actuators including, but not limited to electric, pneumatic, and hydraulic systems. Transmissions may use planetary gearing, fixed-axis belt, chain, cable drives, or linear mechanisms. Passive spring elements can also be incorporated. In certain embodiments, the actuator has a controller that is used to control the power. For example, the controller may be used to start and stop the actuator, regulate the speed, and/or regulate the torque applied to the joint.

The general inventive concept also contemplates methods of modifying or replacing an existing general use orthosis as described herein. In certain embodiments, the methods comprise adapting a passive joint assembly to include one or more attachment features as described above, providing an active joint assembly as described above (and shown e.g., in FIGS. 6-10), and coupling the active joint attachment features of the active joint to the adapted passive joint.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. Unless expressly excluded herein all such combinations and sub-combinations are intended to be within the scope of the present inventions. Still further, while various alternative embodiments as to the various aspects, concepts and features of the inventions, such as alternative materials, structures, configurations, methods, devices and components, alternatives as to form, fit and function, etc. may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions, even if such embodiments are not expressly disclosed herein.

Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated.

While various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention, the inventions instead being set forth in the appended claims. Descriptions of exemplary methods or processes are not limited to inclusion of all steps as being required in all cases, nor is the order that the steps are presented to be construed as required or necessary unless expressly so stated.

1. A device for powered orthosis comprising:
a power source for providing gait power to a limb of an individual;
an adapter for transmitting the gait power from the power source to a passive joint on an orthotic device on a limb of the individual, the orthotic device comprising a rotating member comprising first attachment feature, a non-rotating member comprising a second attachment feature, and a joint connecting the rotating member and the non-rotating member;
the adapter comprising a housing, a first interface feature configured to releasably engage with the first attachment feature on the non-rotating member and a second interface feature configured to releasably engage with second attachment feature on the rotating member;
such that gait power is transmitted from the power source to the joint.

2. The device of claim 1, wherein the orthotic device is a knee-ankle-foot orthotic.

3. The device of claim 1, wherein the orthotic device is a passive joint.

4. The device of claim 1, wherein the adapter is attached at a knee joint of the orthotic device.

5. The device of claim 1, wherein the adapter is attached at a hip joint of the orthotic device.

6. The device of claim 1, wherein the first interface feature is in the shape of a cylinder and the first attachment feature is a cylindrical aperture in the non-rotating member.

7. The device of claim 1, wherein the first interface feature comprises a locking mechanism to maintain relative position between the adapter and the orthotic device.

8. The device of claim 1, wherein the orthotic device comprises an upper cuff adapted to secure around a non-rotating portion of the limb, corresponding to and mechanically engaged to the non-rotating member of the orthotic, and a lower cuff adapted to secure around a rotating portion of the limb, corresponding to and mechanically engaged to the rotating member of the orthotic device.

9. The device of claim 1, wherein the first interface feature is a cylinder and the first attachment feature is a circular aperture in the non-rotating member, wherein the second interface member is an elliptical protrusion and the second attachment means is an elliptical aperture in the rotating member.

10. A method of modifying a general use orthosis, the method comprising
providing an adapter for transmitting the gait power from a power source to a passive joint on the general use orthotic device on a limb of the individual, the orthotic device comprising a rotating member comprising first attachment feature, a non-rotating member comprising a second attachment feature, and a joint connecting the rotating member and the non-rotating member;
the adapter comprising a housing, a first interface feature configured to releasably engage with the first attachment feature on the non-rotating member and a second interface feature configured to releasably engage with second attachment feature on the rotating member;
securing the adapter to the general use orthosis by
engaging the first interface feature with the first attachment feature;
engaging the second interface feature with the second attachment feature; and
engaging the power source with the adapter to deliver the gait power to the passive joint.

11. The device of claim 10, wherein the orthotic device is a knee-ankle-foot orthotic.

12. The method of claim 10, wherein the orthotic device is a passive joint.

13. The method of claim 10, wherein the adapter is attached at a knee joint of the orthotic device.

14. The method of claim 10, wherein the adapter is attached at a hip joint of the orthotic device.

15. The method of claim 10, wherein the first interface feature is in the shape of a cylinder and the first attachment feature is a cylindrical aperture in the non-rotating member.

16. The method of claim 10, wherein the first interface feature comprises a locking mechanism to maintain relative position between the adapter and the orthotic device.

17. The method of claim 10, wherein the orthotic device comprises an upper cuff adapted to secure around a non-rotating portion of the limb, corresponding to and mechanically engaged to the non-rotating member of the orthotic, and a lower cuff adapted to secure around a rotating portion of the limb, corresponding to and mechanically engaged to the rotating member of the orthotic device.

18. The method of claim 10, further comprising engaging a power source in mechanical communication with the adapter to provide power through the adapter to the orthotic device.
